(19) United States
(12) Patent Application Publication
(10) Pub. No.: US 2002/0072078 A1
(43) Pub. Date: Jun. 13, 2002

(54) Rapid test for cell surface antigen

(75) Inventors: Yi-Fen Tsai, Taipei (TW); Liahng-Yirn Liu, Taipei (TW); Hsueh-Fang Chen, Keelung (TW); Tai-Guang Wu, Taipei (TW); Jiuan Liu, Taipei (TW); Tong-Hsuan Chang, Taipei (TW)

Correspondence Address: Y. ROCKY TSAO, Fish & Richardson P.C., 225 Franklin Street, Boston, MA, 02110-2804, US

(73) Assignee: Development Center for Biotechnology, a Taiwan corporation

(21) Appl. No.: 09/988,837

(22) Filed: Nov. 19, 2001

Related U.S. Application Data
(62) Division of application No. 09/560,783, filed on Apr. 28, 2000.

(30) Foreign Application Priority Data
Apr. 28, 1999 (TW) 88106844

(51) Int. Cl. G01N 33/567

(52) U.S. Cl. 435/7.21

(57) ABSTRACT

The present invention discloses a diagnostic kit for assaying a cell presenting specific surface specific antigen, comprising: a first complex, comprising a magnetic bead coated with a first ligand specific to the specific cell surface antigen; a second complex, comprising a second ligand specific to the common cell surface antigen and a signal generation means; and a magnetic support. The kit can be used for the quantitative and qualitative analysis of a cell sample containing specific cell surface antigen.

### BACKGROUND OF THE INVENTION

1. Field of the Invention

The present invention relates to a diagnostic kit and a method for assaying of a sample of cells for presenting a specific cell surface antigen. More particularly, it relates to a diagnostic kit and method for assaying a sample of cells for presenting a specific leukocyte surface antigen.

2. Description of the Related Arts

Cell surface antigens could be classified into two categories: common cell surface antigen and specific cell surface antigen. The former is generally present on the surface of all certain kind of cells (e.g., red blood cells, white blood cells, etc.) whereas the latter is only present individually. It has been noted that the certain specific cell surface antigen is associated to certain diseases. Furthermore, the different races have different specific cell surface antigens. In addition, a higher success rate for implantation can be achieved by matching certain cell specific antigens between the donor and the implant recipient. Due to the polymorphism of human leukocyte antigen (HLA), it has been found to be especially useful in the above-mentioned applications.

Table 1 describes the relationship between human leukocyte antigens and certain diseases. It is known that people with specific surface antigen have higher disease probability than those without this specific surface antigen. One of the examples is ankylosing spondylitis (AS). HLA-B27 has been found to be highly associated with ankylosing spondylitis. The probability of HLA-B27 (one kind of human leukocyte antigens) being present in AS patients is 90%, whereas the probability of it being present in normal people is only 9.4%. In Taiwan, it is estimated that 5.5% of the population bear HLA-B27 gene, and 10-20% of these people may suffer from AS later in their lifetime. Although the relationship between AS and HLA-B27 has not yet been clarified. The HLA-B27 antigen is one of the most effective way to help diagnosis for ankylosing spondylitis.

TABLE 1
A list of the relationship between HLA and diseases

| | | Frequency of HLA examined | |
|---|---|---|---|
| Disease | HLA | Patient (%) | Control (%) |
| Ankylosing Spondylitis | B27 | 90 | 9.4 |
| Reiter's symptom | B27 | 79 | 9.4 |
| Acute anterior uveitis | B27 | 52 | 9.4 |
| Herpes dermatitis | DR3 | 85 | 26.3 |
| Insulin dependent diabetes | DR3, 4 | 91 | 57.3 |
| mellitus | or both | | |
| Narcolepsy | DR2 | 100 | 25.8 |

In addition to the diseases described above, the human leukocyte antigen is related to race. For example, AW43 and BW42 are mostly expressed in Black Africans, while BW46 and BW 54 are mainly expressed in Asians. Therefore, the HLA can be used in race studies.

In the prior art, two methods are used for HLA typing, including microlymphocytotoxicity test (MCLT) and using of the flow cytometry (FC). The MCLT assay is performed via the combination of an antibody against a certain type of HLA thereby causing complement activation. Cells having this type of HLA will be lysed due to cytotoxicity. Since the degree of the lysing is not easy to judge and the judgement is subjective, and the lysing during the operation increase the error of the experiment.

The flow cytometry is performed via an antibody against HLA labeled with fluorochrome. The HLA is examined by the intensity of fluorescence. Therefore, this method is suitable for quantitative and qualitative analysis. The advantage of using of the flow cytometry is simple and convenient; however, the equipment costs more than million NT dollars in addition to the maintenance and materials, this method is comparatively expensive. Another shortcoming is the flow cytometry should be operated by technicians who are specially trained.

Therefore, it is quite challenging and impetuous to develop a rapid, inexpensive and easy-operated detection system for HIA or cell surface antigen.

### SUMMARY OF THE INVENTION

It is therefore a primary object of the present invention is to make improvement for the prior arts described above and provides a diagnostic kit for detecting a cell presenting a specific cell surface antigen, wherein said cell contains a common cell surface antigen. The diagnostic kit comprises (a) a first complex, comprising a magnetic bead coated with a first ligand specific to a specific cell surface antigen; (b) a second complex, comprising a second ligand specific to the common cell surface antigen coupled with a signal generation means; and (c) a magnetic support.

Another objective of the present invention is providing a method of assaying a cell presenting a specific cell surface antigen or monitoring cell's common cell surface antigen with abnormal condition, wherein said cell contains another common cell surface antigen, comprising the steps of: (a) providing a sample containing said cell; (b) providing a first complex, comprising a magnetic bead coated with a first ligand specific to the specific cell surface antigen; (c) providing a second complex, comprising a second ligand specific to the common cell surface antigen coupled with a signal generation means; (d) mixing said sample containing said cell with said first complex and said second complex to form a third complex; (e) providing a magnetic support to immobilize said third complex; (f) separating said third complex from said sample in the presence of said magnetic support; and (g) generating a signal from said third complex.

The present invention will be more fully understood and further advantages will become apparent when reference is made to the following description of the invention.

### DETAILED DESCRIPTION OF THE INVENTION

In the present invention, two complexes are primarily employed to detect the presence of cell surface antigen in a cell sample. In addition to the common cell surface antigen belonging to such kind of cell (e.g., red blood cells, white blood cells, etc.), the cell sample to be analyzed has various specific cell surface antigen according to the individual.

In accordance with the present invention, the first complex comprises the magnetic bead coated with the first ligand specific to the specific cell surface antigen, such that the cells having this antigen are immobilized from aqueous solution. The first ligand is an antibody which is specific to the specific cell surface antigen, and more preferably, the first ligand is a monoclonal antibody. For the purpose of convenient operation, the magnetic bead used herein is a particle with magnetism and the diameter is about 1-5 microns. The magnetic bead is made of suitable magnetic materials and has the advantage of extremely small volume and thus does not affect the reaction hereinafter. Examples of suitable magnetic material include, for example, ferrite, perovskite or chromite. In one preferred embodiment, the specific cell surface antigen is human leukocyte specific antigen B27.

The method for producing the first complex comprises incubating the first ligand with the magnetic bead in a solution and mixing well for a period of time. The first ligand then coated onto the surface of the magnetic bead to form the first complex. Because the first ligand coated onto the magnetic bead is specific to the specific cell surface antigen, the first complex can react with the specific cell surface antigen. The solution used herein may be, for example, aqueous solution or saline buffer solution.

The second complex is responsible for the signal generation, from which the analyzer can judge the presence of cells having the specific cell surface antigen, or monitor the expression level of the specific cell surface antigen. The second complex comprises the second ligand specific to the common cell surface antigen coupled with a signal generation means. The common cell surface antigen may be any one of the common cell surface antigen. In one preferred embodiment, the common cell surface antigen is human leukocyte common antigen CD45. The second ligand is an antibody which is specific to the common cell surface antigen, and more preferably, the second ligand is a monoclonal antibody. The signal generation means can include, but is not limited to, radioactive material, fluorescent material, luminescent material or enzymes. Enzymes which can react with a certain substrate for development include, but are not limited to, horseradish peroxidase (HRPO), hydrogen peroxidase, alkaline phosphatase, β-galactosidase and glucose oxidase. Thus, the diagnostic kit of the present invention may further comprise a substrate reacting with the enzyme to generate a signal.

According to the detection method of the present invention, a sample (e.g. blood sample) of cells containing the cell surface antigen is placed in a container, and then the first and second complexes are added. The container used herein is made of non-magnetic materials, thus the combination of the cells to be examined and ligands described above will not be affected.

After mixing for a period of time, the first complex having the first ligand binds to the cells having the specific cell surface antigen, but does not bind to those without the antigen.

Afterwards, a magnetic support provides magnetic field to immobilize the cells which have been bound to the first complex. On the other hand, the cells without the specific cell surface antigen are still homogeneously suspended in the solution. Then, the sample is washed with a solution several times to thoroughly remove the unbound second complex and cells without the specific cell surface antigen in the presence of the magnetic support. The magnetic support used herein for the immobilization of the magnetic beads includes, for example, a magnetic rack or plate. Solutions that can be used for washing sample without destroying cells or reacting with the components include normal saline, medium or buffer solution.

After removing the unbound second complex and cells without the specific cell surface antigen, the magnetic support is also removed. Then a signal is generated via the signal generation means. Thus, the presence of the specific cell surface antigen in the sample or the amount thereof can be estimated by way of the intensity of the signal. The signal generation means used herein includes, for example, a substrate which can be reacted with the enzyme for development. Other signal generation means are described above.

Without intending to limit it in any manner, the present invention will be further illustrated by the following preferred examples. The specific cell surface antigen used in the following preferred examples is human leukocyte specific antigen B27, and the common cell surface antigen used is human leukocyte common antigen CD45.

### EXAMPLE

### Example 1

### Process for Using 96-Well Microtiter Plate (ELISA)

100 μl of whole blood containing HLA-B27 of two individuals and another two without HLA-B27 were added into the 96-well microtiter plate, respectively. 10 μl of 2.5 mg/ml magnetic beads coated with anti HLA-B27 monoclonal antibody, and 10 μl of 5 μg/ml antibody against CD45 coupled with horseradish peroxidase (HRPO) were added to the wells described above at room temperature for 20 minutes. A magnetic plate was then placed at the bottom of the 96-well microtiter plate to immobilize the magnetic beads. The microtiter plate was washed with 250 μl of phosphate buffered saline four times and the supernatant was then pipetted out. 100 μl of peroxidase substrate TMB (3,3′,5,5′-tetramethylbensidine) was added at room temperature for 5 minutes, followed by the addition of 50 μl of 2 M HCl for stopping the reaction. Finally, the absorbance of the microtiter plate was read at 450 nm by ELISA reader. The result is shown in Table 2.

TABLE 2

| | Sample containing HLA-B27 | | Sample without HLA-B27 | |
|---|---|---|---|---|
| Individual | 1 | 2 | 1 | 2 |
| Absorbance | 1.168 | 1.281 | 0.084 | 0.093 |
| Absorbance | 1.237 | 2.611 | 0.518 | 0.689 |

### Example 2

### Process for Using Test Tube

100 μl of whole blood containing HLA-B27 and whole blood without HLA-B27 of two individuals were added into a test tube, respectively. 10 μl of 2.5 mg/ml magnetic beads coated with HLA-B27 monoclonal antibody, and 10 μl of 5 μg/ml antibody against CD45 which is coupled with horseradish peroxidase (HRPO) were added to the tubes described above at room temperature for 15 minutes. The magnetic rack was then placed beside the test tubes to immobilize the magnetic beads. The test tubes were thrice washed with 1 ml of phosphate buffered saline and the supernatant was then pipetted out. 200 μl of peroxidase substrate TMB (3,3′,5,5′-tetramethylbensidine) was added at room temperature for 5 minutes, followed by the addition of 50 μl of 2 M HCl for stopping the reaction. Finally, the absorbance of the supernatant was read at 450 nm by spectrophotometer. The result is shown in Table 3.

TABLE 3

| | Sample containing HLA-B27 | | Sample without HLA-B27 | |
|---|---|---|---|---|
| Individual | 1 | 2 | 1 | 2 |
| Absorbance | 1.655 | 1.570 | 0.589 | 0.915 |

While the invention has been particularly shown and described with the reference to the preferred embodiment thereof, it will be understood by those skilled in the art that various changes in form and details may be made without departing from the spirit and scope of the invention.

What is claimed is:

1. A diagnostic kit for assaying a cell presenting a specific cell surface antigen, wherein said cell contains a common cell surface antigen, comprising:
(a) a first complex, comprising a magnetic bead coated with a first ligand specific to the specific cell surface antigen;
(b) a second complex, comprising a second ligand specific to the common cell surface antigen coupled with a signal generation means; and
(c) a magnetic support.

2. The diagnostic kit as claimed in claim 1, wherein said first ligand is a monoclonal antibody.

3. The diagnostic kit as claimed in claim 1, wherein said second ligand is a monoclonal antibody.

4. The diagnostic kit as claimed in claim 1, wherein the diameter of said magnetic bead is about 1-5 microns.

5. The diagnostic kit as claimed in claim 1, wherein the material of said magnetic bead is selected from the group consisting of ferrite, perovskite and chromite.

6. The diagnostic kit as claimed in claim 1, wherein said signal generation means is selected from the group consisting of radioactive material, fluorescent material, luminescent material and enzymes.

7. The diagnostic kit as claimed in claim 6, wherein said enzyme is selected from the group consisting of horseradish peroxidase, hydrogen peroxidase, alkaline phosphatase, β-galactosidase and glucose oxidase.

8. The diagnostic kit as claimed in claim 7, further comprising a substrate operatively reacted with said enzyme to generate a signal.

9. The diagnostic kit as claimed in claim 1, wherein said magnetic support is a magnetic material, which provides magnetic field.

10. A method of assaying a cell presenting a specific cell surface antigen, wherein said cell contains a common cell surface antigen, comprising the steps of:
(a) providing a sample containing said cell;
(b) providing a first complex, comprising a magnetic bead coated with a first ligand specific to the specific cell surface antigen;
(c) providing a second complex, comprising a second ligand specific to the common cell surface antigen coupled with and a signal generation means;
(d) mixing said sample containing said cell with said first complex and said second complex to form a third complex;
(e) providing a magnetic support to immobilize said third complex;
(f) separating said third complex from said sample in the presence of said magnetic support; and
(g) generating a signal from said third complex.

11. The method as claimed in claim 10, wherein said first ligand is a monoclonal antibody.

12. The method as claimed in claim 10, wherein said second ligand is a monoclonal antibody.

13. The method as claimed in claim 10, wherein the material of said magnetic bead is selected from the group consisting of ferrite, perovskite and chromite.

14. The method as claimed in claim 10, wherein the diameter of said magnetic bead is about 1-5 microns.

15. The method as claimed in claim 10, wherein said signal generation means is selected from the group consisting of radioactive material, fluorescent material, luminescent material and enzymes.

16. The method as claimed in claim 15, wherein said enzyme is selected from the group consisting of horseradish peroxidase, hydrogen peroxidase, alkaline phosphatase, β-galactosidase and glucose oxidase.

17. The method as claimed in claim 10, wherein the signal in step (g) is generated by reacting said enzyme with a substrate.

18. The method as claimed in claim 10, further comprising the steps of washing said sample with a buffer and then removing the buffer in step (f).

19. The method as claimed in claim 10, wherein said magnetic support is a magnetic material, which provides magnetic field.

20. The method as claimed in claim 10, wherein said cell is human leukocyte.

21. The method as claimed in claim 20, wherein the specific cell surface antigen is human leukocyte antigen B27.

22. The method as claimed in claim 21, wherein the common cell surface antigen is human leukocyte antigen CD45.
